(19) United States
(12) Patent Application Publication
(10) Pub. No.: US 2014/0065096 A1
(43) Pub. Date: Mar. 6, 2014

(54) CANCER THERAPY BY EX VIVO ACTIVATED AUTOLOGOUS IMMUNE CELLS

(71) Applicant: Regen BioPharma, Inc., La Mesa, CA (US)

(72) Inventors: Thomas Ichim, San Diego, CA (US); David Koos, La Mesa, CA (US)

(73) Assignee: Regen BioPharma, Inc., La Mesa, CA (US)

(21) Appl. No.: 13/957,431

(22) Filed: Aug. 2, 2013

Related U.S. Application Data
(60) Provisional application No. 61/697,025, filed on Sep. 5, 2012.

(51) Int. Cl. A61K 35/14; A61K 45/06; A61K 31/375; A61K 38/20; A61K 38/21

(52) U.S. Cl. CPC A61K 35/17; A61K 38/20; A61K 38/212; A61K 38/215; A61K 38/217; A61K 31/375; A61K 38/2006; A61K 38/2013; A61K 38/2046; A61K 38/208; A61K 38/2086; A61K 45/06
USPC 424/85.2; 424/184.1; 424/234.1; 424/248.1; 424/85.7; 424/85.6; 424/85.5; 514/474; 424/178.1

(57) ABSTRACT

Disclosed are therapeutic methods for ex-vivo activation of immune cells from a cancer patient for the purpose of inducing tumor regression and/or suppressing metastasis and/or tumor recurrence. In one embodiment mononuclear cells of a patient are isolated from peripheral blood and activated by a combination of innate immune system activators together with means allowing for T cell activation.

### CROSS-REFERENCE TO RELATED APPLICATIONS

This application claims priority to U.S. Provisional Application No. 61/697,025, filed Sep. 5, 2012, and entitled “TREATMENT OF NEOPLASIA USING AUTOLOGOUS ACTIVATED IMMUNOCYTES”, which is hereby expressly incorporated by reference in its entirety.

### FIELD OF THE INVENTION

The invention pertains to the area of immune modulation, specifically to the area of treating cancer using the immune system of the patient. More specifically, the invention pertains to the area of adoptive immunotherapy.

### BACKGROUND

Surgery, radiation therapy, and chemotherapy have been the standard accepted approaches for treatment of cancers including leukemia, solid tumors, and metastases. Unfortunately, these approaches are associated with extremely high toxicity and adverse effects. Immunotherapy which uses the body's immune system, either directly or indirectly, to shrink or eradicate cancer has been studied for many years as an adjunct to conventional cancer therapy. It is believed that the human immune system is an untapped resource for cancer therapy and that effective treatment can be developed once the components of the immune system are properly harnessed. As key immunoregulatory molecules and signals of immunity are identified and prepared as therapeutic reagents, the clinical effectiveness of such reagents can be tested using established cancer models. Immunotherapeutic strategies include administration of vaccines, activated cells, antibodies, cytokines, chemokines, as well as small molecular inhibitors, anti-sense oligonucleotides, and gene therapy. It is believed by many that immunotherapy offers the potential for treatment of cancer without the toxicities associated with current approaches to cancer therapy.

The current focus of cancer research in general is the creation of therapies that not only destroy, inhibit, or block progression of primary tumors, but also suppress micrometastatic and metastatic progeny of the primary tumor from seeding the patient. Despite extensive research into the disease, effective means of treating the majoring of cancers at present have not been developed by the medical community. Although limited success is achieved using the current standard therapies: chemotherapy, radiation therapy, and surgery; each therapy has its own inherent limitations. Chemotherapy and radiation therapy have devastating consequences causing extensive damage to normal, healthy tissue such as bone marrow, intestinal cells, and neuronal cells, despite efforts to target such therapy to abnormal tissue (e.g., tumors). Surgery is in many cases effective in removing masses of cancerous cells; however, it cannot always ensure complete removal of affected tissue nor are all tumors in an anatomical location amenable to surgical removal. Furthermore seeding of distant tissues by the excised tumor during the process of removal or beforehand are significant problems.

Immunological control of neoplasia, specifically the ability of the immune system to control cancer, is suggested by evidence of longer survival of patients with a variety of cancers who possess a high population of tumor infiltrating lymphocytes [1-3]. Additionally, the observation has been made that immune suppressed patients, either as a result of transplant immune suppression or genetic conditions, develop cancer at a much higher frequency in comparison to non-immune suppressed individuals [4, 5]. Additionally, some data supports the notion that in some situations immunotherapy of cancer is effective [6]. While cancer immunotherapy offers the possibility of inducing remission and control of both the primary tumor mass, as well as micrometastasis, several drawbacks exist. The most significant one is that in many situations immunotherapy is either not powerful enough to cause a significant reduction of tumors, or is associated with a variety of toxicities.

Various types of immunotherapies for cancer have been tried, including: a) systemic cytokine administration; b) gene therapy; c) allogeneic vaccines; d) autologous vaccine; e) heat shock protein vaccines; f) dendritic cell vaccines; g) tumor infiltrating lymphocytes; h) administration of T cells in a lymphodepleted environment; and i) nutritional interventions. Although each of the approaches contains significant advantages and drawbacks, none of them simultaneously meet the criteria of reproducible efficacy, availability to the mass population, or specificity. The one exception to this is autologous PAP-GM-CSF pulsed dendritic cells developed by the company Dendreon.

Another type of immunotherapy is the use of systemically acting immune stimulants such as interleukin-2 (IL-2). The precursor of such therapies actually began with the work of William Coley who induced a systemic inflammatory/immune activation through administration of killed S pyogenes and Serratia marcescens bacteria in patients with soft tissue sarcoma [7]. The advent of molecular biology allowed for assessment of molecular signals associated with systemic immune activation. The cytokine tumor necrosis factor (TNF)-alpha was one of the molecular signals associated with anticancer efficacy of innate immune activators such as the Coley vaccine [8]. Studies have demonstrated that TNF-alpha has the ability to induce profound death of cancer cells in vitro and in vivo in animal models, however human studies demonstrated unacceptable levels of toxicity [9-11]. IL-2 was the next cytokine associated with immune activation that was tested. Originally termed T Cell Growth Factor (TCGF) [12], IL-2 was demonstrated in early studies to endow human lymphocytes with ability to selectively kill tumor but not healthy control cells [13]. Subsequent studies have demonstrated that cytotoxic activity was mediated through T cell and natural killer (NK) cells, whose activation requires stimulation of the IL-2 receptor [14, 15], which can be accomplished in vivo with high doses of IL-2 [16-18]. Animal studies suggested that IL-2 has a short half-life of approximately 2 minutes after intravenous injection [19, 20], and human half life was reported to be approximately an hour [21]. Thus it was apparent that clinical use of IL-2 would be requiring repeated administration at high doses. Despite this pitfall, preclinical studies demonstrated highly potent anti-tumor effect. In 1985 Steven Rosenberg reported regression of established pulmonary metastasis, as well as various subcutaneous tumors by administration of IL-2 [22]. These data were highly promising due to the fact that tumor killing could be achieved systemically, and by activation of specific immune cells that could be identified in vivo as interacting with and inducing death of the tumor.

Early studies of IL-2 demonstrated impressive results in a subset of melanoma and renal cell cancer patients. These studies were expanded and eventually IL-2 received approval as the first recombinant immunotherapeutic drug by the FDA. There appears to be a dose response with IL-2 in that the doses that seem to be most effective are also associated with significant toxicity. The most significant cause of toxicity is vascular leak syndrome (VSL), manifested as fluid loss into the interstitial space, which is a result of increase vessel permeability. Additional effects include thrombocytopenia, elevated hepatic serum transaminases, hepatocyte necrosis, hypoalbuminemia, tissue and peripheral eosinophilia, and prerenal azotemia [23].

Thus it is apparent that the limitations of many immunotherapeutic approaches to cancer is that tumor antigens are either not clearly defined, or in situations where they are defined, the tumor either mutates to lose expression of such antigens, or the antigen-specific vaccine is only applicable to patients with a certain major histocompatibility complex haplotype. The circumvention of this problem has been attempted using autologous vaccines, however in many cases this is an expensive and difficult procedure.

### SUMMARY

Embodiments herein are directed to methods of treating cancer comprising: a) culturing autologous mononuclear cells derived from an autologous source; b) treating said mononuclear cells with an agent activating innate immune cells found in said mononuclear cell population; and c) re-administering activated immune cells into the same patient.

### DETAILED DESCRIPTION

In one embodiment the invention provides a means of generating a population of cells with tumoricidal ability. Peripheral blood is extracted from a cancer patient and peripheral blood monoclear cells (PBMC) are isolated using the Ficoll Method. PBMC are subsequently resuspended in 10 ml STEM-34 media and allowed to adhere onto a plastic surface for 2-4 hours. The adherent cells are then cultured at 37° C. in STEM-34 media supplemented with 1,000 U/mL granulocyte-monocyte colony-stimulating factor and 500 U/mL IL-4 after non-adherent cells are removed by gentle washing in Hanks Buffered Saline Solution (HBSS). Half of the volume of the GM-CSF and IL-4 supplemented media is changed every other day. Immature DCs are harvested on day 7. In one embodiment said generated DC are used to stimulate T cell and NK cell tumoricidal activity. Incubation with interferon gamma may be performed for the period of 2 hours to the period of 7 days. Preferably, incubation is performed for approximately 24 hours, after which T cells and/or NK cells are stimulated via the CD3 and CD28 receptors. One means of accomplishing this is by addition of antibodies capable of activating these receptors. In one embodiment approximately, 2 ug/ml of anti-CD3 antibody is added, together with approximately 1 ug/ml anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 500 u/mL IL-2. Media containing IL-2 and antibodies may be changed every 48 hours for approximately 8-14 days. In one particular embodiment DC are included to said T cells and/or NK cells in order to endow cytotoxic activity towards tumor cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or into an afferent lymph vessel.

In some embodiments, the culture of the cells is performed by starting with purified lymphocyte populations, for example, The step of separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4, and separation methods depending on these surface markers are known in the art. For example, the step can be performed by mixing a carrier such as beads or a culturing container on which an anti-CD8 antibody has been immobilized, with a cell population containing a T cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as an index and, for example, CD4 MicroBeads, Dynabeads M-450 CD4 can also be used. In some embodiments of the invention, T regulatory cells are depleted before initiation of the culture. Depletion of T regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-.gamma., transforming growth factor (TGF)-.beta., IL-15, IL-7, IFN-.alpha., IL-12, CD40L, and IL-27. From the viewpoint of enhancing cellular immunity, particularly suitably, IL-2, IFN-.gamma., or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1.alpha., MIP1.beta., CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects.

In one embodiment of the invention, ascorbic acid is administered intravenously together with activated lymphocytes which possess tumor inhibitory/killing activity. In a preferred embodiment the intravenous vitamin C is administered once every two days at a concentration of 10 g per injection. The rational for use of intravenous vitamin C comes from observations of a scurvy-like condition in a renal cell carcinoma patient treated with IL-2. The patient presented with acute signs and symptoms of scurvy (perifollicular petechiae, erythema, gingivitis and bleeding). Serum ascorbate levels were significantly reduced to almost undetectable levels [24]. Although the role of ascorbic acid (AA) hypersupplementation in stimulation of immunity in healthy subjects is controversial, it is well established that AA deficiency is associated with impaired cell mediated immunity. This has been demonstrated in numerous studies showing deficiency suppresses T cytotoxic responses, delayed type hypersensitivity, and bacterial clearance [25]. Additionally, it is well-known that NK activity, which IL-2 is anti-tumor activity is highly dependent on, is suppressed during conditions of AA deficiency [26]. Thus it may be that while IL-2 therapy on the one hand is stimulating T and NK function, the systemic inflammatory syndrome-like effects of this treatment may actually be suppressed by induction of a negative feedback loop. Such a negative feedback loop with IL-2 therapy was successfully overcome by work using low dose histamine to inhibit IL-2 mediated immune suppression, which led to the “drug” Ceplene (histamine dichloride) receiving approval as an IL-2 adjuvant for treatment of AML [27].

The concept of AA deficiency subsequent to IL-2 therapy (as an example of an immune stimulatant) was reported previously by another group. Marcus et al evaluated 11 advanced cancer patients suffering from melanoma, renal cell carcinoma and colon cancer being on a 3 phase immunotherapeutic program consisting of: a) 5 days of i.v. high-dose (10(5) units/kg every 8 h) interleukin 2, (b) 6½ days of rest plus leukapheresis; and (c) 4 days of high-dose interleukin 2 plus three infusions of autologous lymphokine-activated killer cells. Mean plasma ascorbic acid levels were normal (0.64+/−0.25 mg/dl) before therapy. Mean levels dropped by 80% after the first phase of treatment with high-dose interleukin 2 alone (0.13+/−0.08 mg/dl). Subsequently plasma ascorbic acid levels remained severely depleted (0.08 to 0.13 mg/dl) throughout the remainder of the treatment, becoming undetectable (less than 0.05 mg/dl) in eight of 11 patients during this time. Importantly, blood pantothenate and plasma vitamin E remained within normal limits in all 11 patients throughout the phases of therapy, suggesting the hypovitaminosis was specific AA. Strikingly, Responders (n=3) differed from nonresponders (n=8) in that plasma ascorbate levels in the former recovered to at least 0.1 mg/dl (frank clinical scurvy) during Phases 2 and 3, whereas levels in the latter fell below this level [28]. Similar results were reported in another study by the same group examining an additional 15 patients [29]. The possibility that prognosis was related to AA levels is strongly suggested.

One skilled in the art will appreciate that these methods and devices are and can be adapted to carry out the objects and obtain the ends and advantages mentioned, as well as those inherent therein. The methods, procedures, and devices described herein are presently representative of preferred embodiments and are exemplary and are not intended as limitations on the scope of the invention. Changes therein and other uses will occur to those skilled in the art which are encompassed within the spirit of the invention and are defined by the scope of the disclosure.

It is apparent to one skilled in the art that varying substitutions and modifications can be made to the invention disclosed herein without departing from the scope and spirit of the invention. Furthermore, those skilled in the art recognize that the aspects and embodiments of the invention set forth herein can be practiced separate from each other or in conjunction with each other. Therefore, combinations of separate embodiments are within the scope of the invention as disclosed herein.

### BIBLIOGRAPHY

1. Ryschich, E., et al., Control of T-cell-mediated immune response by HLA class I in human pancreatic carcinoma. Clin Cancer Res, 2005. 11(2 Pt 1): p. 498-504.
2. Raspollini, M. R., et al., Tumour-infiltrating gamma/delta T-lymphocytes are correlated with a brief disease-free interval in advanced ovarian serous carcinoma. Ann Oncol, 2005. 16(4): p. 590-6.
3. Chiba, T., et al., Intraepithelial CD8+ T-cell-count becomes a prognostic factor after a longer follow-up period in human colorectal carcinoma: possible association with suppression of micrometastasis. Br J Cancer, 2004. 91(9): p. 1711-7.
4. Astigiano, S., et al., Eosinophil granulocytes account for indoleamine 2,3-dioxygenase-mediated immune escape in human non-small cell lung cancer. Neoplasia, 2005. 7(4): p. 390-6.
5. Whiteside, T. L., Down-regulation of zeta-chain expression in T cells: a biomarker of prognosis in cancer? Cancer Immunol Immunother, 2004. 53(10): p. 865-78.
6. Rosenberg, S. A. and M. E. Dudley, Cancer regression in patients with metastatic melanoma after the transfer of autologous antitumor lymphocytes. Proc Natl Acad Sci USA, 2004. 101 Suppl 2: p. 14639-45.
7. Hoption Cann, S. A., J. P. van Netten, and C. van Netten, Dr William Coley and tumour regression: a place in history or in the future. Postgrad Med J, 2003. 79(938): p. 672-80.
8. Wiemann, B. and C. O. Starnes, Coley's toxins, tumor necrosis factor and cancer research: a historical perspective. Pharmacol Ther, 1994. 64(3): p. 529-64.
9. Balkwill, F., Tumour necrosis factor and cancer. Nat Rev Cancer, 2009. 9(5): p. 361-71.
10. Mueller, H., Tumor necrosis factor as an antineoplastic agent: pitfalls and promises. Cell Mol Life Sci, 1998. 54(12): p. 1291-8.
11. Taguchi, T. and Y. Sohmura, Clinical studies with TNF. Biotherapy, 1991. 3(2): p. 177-86.
12. Ruscetti, F. W. and R. C. Gallo, Human T-lymphocyte growth factor: regulation of growth and function of T lymphocytes. Blood, 1981. 57(3): p. 379-94.
13. Lotze, M. T., et al., Lysis of fresh and cultured autologous tumor by human lymphocytes cultured in T-cell growth factor. Cancer Res, 1981. 41(11 Pt 1): p. 4420-5.
14. Robb, R. J., A. Munck, and K. A. Smith, T cell growth factor receptors. Quantitation, specificity, and biological relevance. J Exp Med, 1981. 154(5): p. 1455-74.
15. Miyawaki, T., et al., Functional significance of Tac antigen expressed on activated human T lymphocytes: Tac antigen interacts with T cell growth factor in cellular proliferation. J Immunol, 1982. 129(6): p. 2474-8.
16. Eberlein, T. J., et al., Immunomodulatory effects of systemic low-dose recombinant interleukin-2 and lymphokine-activated killer cells in humans. Cancer Immunol Immunother, 1989. 30(3): p. 145-50.
17. Ting, C. C., M. E. Hargrove, and D. Stephany, Generation of activated killer cells in tumor-bearing hosts. Int J Cancer, 1987. 39(2): p. 232-9.
18. Fletcher, M. and A. L. Goldstein, Recent advances in the understanding of the biochemistry and clinical pharmacology of interleukin-2. Lymphokine Res, 1987. 6(1): p. 45-57.
19. Chang, A. E., C. L. Hyatt, and S. A. Rosenberg, Systemic administration of recombinant human interleukin-2 in mice. J Biol Response Mod, 1984. 3(5): p. 561-72.
20. Donohue, J. H. and S. A. Rosenberg, The fate of interleukin-2 after in vivo administration. J Immunol, 1983. 130(5): p. 2203-8.
21. Cheever, M. A., et al., Interleukin 2 (IL 2) administered in vivo: influence of IL 2 route and timing on T cell growth. J Immunol, 1985. 134(6): p. 3895-900.
22. Rosenberg, S. A., et al., Regression of established pulmonary metastases and subcutaneous tumor mediated by the systemic administration of high-dose recombinant interleukin 2. J Exp Med, 1985. 161(5): p. 1169-88.
23. Mier, J. W., et al., Toxicity of immunotherapy with interleukin-2 and lymphokine-activated killer cells. Pathol Immunopathol Res, 1988. 7(6): p. 459-76.
24. Alexandrescu, D. T., C. A. Dasanu, and C. L. Kauffman, Acute scurvy during treatment with interleukin-2. Clin Exp Dermatol, 2009. 34(7): p. 811-4.
25. Anthony, H. M. and C. J. Schorah, Severe hypovitaminosis C in lung-cancer patients: the utilization of vitamin C in surgical repair and lymphocyte-related host resistance. Br J Cancer, 1982. 46(3): p. 354-67.
26. McMurray, D. N., Cell-mediated immunity in nutritional deficiency. Prog Food Nutr Sci, 1984. 8(3-4): p. 193-228.
27. http://www.highbeam.com/doc/1G1-186526887.html.
28. Marcus, S. L., et al., Severe hypovitaminosis C occurring as the result of adoptive immunotherapy with high-dose interleukin 2 and lymphokine-activated killer cells. Cancer Res, 1987. 47(15): p. 4208-12.
29. Marcus, S. L., et al., Hypovitaminosis C in patients treated with high-dose interleukin 2 and lymphokine-activated killer cells. Am J Clin Nutr, 1991. 54(6 Suppl): p. 1292S-1297S.

1. A method of treating cancer comprising of: a) culturing autologous mononuclear cells derived from an autologous source; b) treating said mononuclear cells with an agent activating innate immune cells found in said mononuclear cell population; and c) re-administering activated immune cells into the same patient.

2. The method of claim 1, wherein an antioxidant is added.

3. The method of claim 2, wherein said antioxidant is selected from a group derived from: a) n-acetylcysteine; b) superoxide dismutase; c) resveratrol; and e) ascorbic acid.

4. The method of claim 3, said antioxidant is administered intravenously.

5. The method of claim 4, wherein ascorbic acid is administered at a concentration ranging from 5 grams to 50 grams intravenously into a 70 kg patient.

6. The method of claim 5, wherein ascorbic acid is administered intravenously at a concentration of 10 grams intravenously into a 70 kg patient.

7. The method of claim 6, wherein said antioxidant is administered at a concentration sufficient to induce inhibition of tumor growth.

8. The method of claim 6, wherein said ascorbic acid administered once per week.

9. The method of claim 1, wherein said agent capable of stimulating activation of cells of the innate immune system is selected from a group comprising of: BCG, imiqimod, beta-glucan, hsp65, hsp90, HMGB-1, lipopolysaccharide, Pam3CSK4, Poly I: Poly C, Flagellin, MALP-2, Imidazoquinoline Resiquimod, CpG oligonucleotides, zymosan, peptidoglycan, lipoteichoic acid, lipoprotein from gram-positive bacteria, lipoarabinomannan from mycobacteria, Polyadenylic-polyuridylic acid, monophosphoryl lipid A, single stranded RNA, double stranded RNA, 852A, rintatolimod, Gardiquimod, and lipopolysaccharide peptides.

10. The method of claim 9, wherein said activator of innate immune system cells is an activator of NF-kappa B.

11. The method of claim 9, wherein said innate immune system activator causes a substantial reduction in phagocytic activity of said dendritic cell after treatment with said stimulator of maturation as compared to before treatment with said stimulator.

12. The method of claim 1, wherein said agents that activate cells of the adaptive immune system are selected from a group comprising of: a cytokine; an agonist of the T cell receptor; an agonist of a costimulatory receptor; an inhibitor of a co-inhibitory molecule.

13. The method of claim 12, wherein said cytokine capable of activating said cells of the adaptive immune system are selected from a group comprising of: IL-1, IL-2, IL-7, IL-12, IL-15, IL-17, IL-21, IL-22, IL-30, IL-33, interferon alpha, interferon beta, interferon gamma, TRANCE, TAG-7, CEL-1000, and LIGHT.

14. The method of claim 12, wherein said agonist of said T cell receptor is selected from a group comprising of: a lectin, an anti-CD3 antibody, a peptide, a peptide ligand, an altered peptide ligand, an agonistic peptide, an agonistic aptamer, and a crosslinking chemical moiety.

15. The method claim 12, wherein said activation of said T cell receptor is accomplished by exposing said T cells to a solid substrate containing anti-CD3 antibodies that have been immobilized to said solid surface.

16. A method for decreasing toxicity of an immunotherapeutic comprising of: a) administering said immunotherapeutic; and b) administering an antioxidant.

17. The method of claim 16, wherein said immunotherapeutic is interleukin-2.

18. The method of claim 16, wherein said antioxidant is intravenous ascorbic acid.

19. The method of claim 18, wherein said intravenous ascorbic acid is administered at a concentration of 5-50 grams per treatment, with 1 treatment per week.

20. The method of claim 18, wherein said intravenous ascorbic acid is administered at a concentration of 10 grams per treatment, with 1 treatment per week.
